(19) United States
(12) Patent Application Publication
(10) Pub. No.: US 2020/0306078 A1
(43) Pub. Date: Oct. 1, 2020

(54) SELF-ADHERING THERAPY PACK

(71) Applicant: MICHAEL N. SOLTES, SEATTLE, WA (US)

(72) Inventor: MICHAEL N. SOLTES, SEATTLE, WA (US)

(21) Appl. No.: 16/901,593

(22) Filed: Jun. 15, 2020

Related U.S. Application Data
(63) Continuation of application No. 15/590,692, filed on May 9, 2017.
(60) Provisional application No. 62/357,837, filed on Jul. 1, 2016.

(51) Int. Cl. A61F 7/02; B32B 37/26; B32B 27/36; B32B 27/08; B32B 5/24; B32B 27/12; B32B 27/30; B32B 27/32; B32B 7/06; B32B 27/06; B32B 27/34; B32B 5/02; B32B 5/32; B32B 7/12

(52) U.S. Cl. CPC A61F 7/02; B32B 2260/021; B32B 27/36; B32B 27/08; B32B 5/24; B32B 27/12; B32B 27/304; B32B 27/32; B32B 7/06; B32B 27/065; B32B 5/245; B32B 27/34; B32B 5/022; B32B 5/32; B32B 7/12; B32B 2037/268; B32B 2310/14; B32B 2255/02; B32B 2367/00; B32B 2535/00; A61F 2007/0219; A61F 2007/0226; A61F 2007/0244; A61F 2007/0261; B32B 2307/728; B32B 2262/0253; B32B 2250/40; B32B 2266/122; B32B 2307/748; B32B 2307/732; B32B 2307/54; B32B 37/26

(57) ABSTRACT

A therapy pack system provides a reusable self-adhering therapy pack to skin. The therapy pack may be for example, an ice pack, heat pack or hot and cold pack. A sticky polymer gel is mounted onto a polymer casing of the therapy pack. The polymer gel is re-usable to attach the therapy pack to a user's skin at the site needing therapy. The polymer gel also buffers the skin from heat or freezer burn associated with applying a hot/cold pack to skin for an extended period. A chloride based compound may be added to the polymer gel to prevent the gel from freezing. In some embodiments, a non-woven fabric patch may support the polymer gel adhesive providing an additional layer of protection to the skin while simultaneously allowing the gel to self-adhere the therapy pack to the user.

### CROSS-REFERENCE TO RELATED APPLICATION

This application claims benefit of priority under 35 U.S.C. § 119 of U.S. Non-Provisional application Ser. No. 15/590,692, filed May 9, 2017, which claims benefit of priority to U.S. Provisional Application No. 62/357,837 filed Jul. 1, 2016, which are hereby incorporated by reference herein in their entirety.

### FIELD

The subject disclosure relates to therapeutic devices, and more particularly, to a self-adhering therapy pack.

### BACKGROUND

A traditional therapy pack is inconvenient, requires that one hold the pack manually to an area or secures the pack by way of strap or tape.

For example, athletes are known to use adhesive tape or saran wrap to strap bags of ice to areas of the body. Not only is the ice rigid and uncomfortable to the skin but repositioning is impractical, circulation is impaired and removing the ice bags subjects the skin to the pain of the removal of the tape on some portion of the skin.

There are areas that cannot be reasonably strapped (for example, the neck, chest, glutes, back) and as such, a user would need to hold a pack for 7-10 minutes, or press against an object (for example, a wall, fridge, door) to hold the pack steady. As will be appreciated, this can present quite an inconvenience to the user as one's hands become tired holding the pack in place. When using ice packs, the hands are exposed to unnecessary cold for a prolonged period. Moreover, the hands prematurely warm the pack, which means the area being healed receives less cold therapy.

Some current therapy packs, require straps, hook and loop systems, snaps or sewn enclosures to secure the pack against skin. Hook and loop systems typically require wrapping a strap around a limb to secure the pack. These methods are difficult to self-administer, can cut off circulation and are limited to areas where a strap can be wound around a limb (arm, leg, etc.).

As can be seen, there is a need for a device that delivers hot or cold therapy treatment that can self-adhere to the skin.

### SUMMARY

In one aspect of the disclosure, a self-adhering therapy pack system comprises a hot/cold gel based therapy pack comprising a polymer casing adapted for use in hot or cold type therapy. A layer of sticky polymer gel adhesive is coupled to an exterior surface of the polymer casing by a tie layer. In addition, a coated-polyester cover is included that is re-attachable to the layer of sticky polymer gel and re-usable to cover the layer of sticky polymer gel.

In another aspect, a self-adhering therapy pack system comprises a hot/cold therapy pack comprising a gel housed in a polymer casing adapted for use in hot or cold type therapy. A first layer of acrylate adhesive is included, wherein a first side of the first layer of acrylate adhesive is coupled to an exterior surface of the polymer casing. A layer of polyester rigidizer non-woven fabric is attached to a second side of the first layer of acrylate adhesive. A layer of acrylate adhesive may be positioned between the exterior surface of the polymer casing and the layer of polyester rigidizer non-woven fabric. A layer of hydrogel is attached to layer of polyester rigidizer non-woven fabric. The system additionally includes a polyester cover re-attachable to the layer of hydrogel and re-usable to cover the layer of hydrogel.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 is a top view of a self-adhering therapy pack system according to an embodiment of the subject technology.

FIG. 2 is a bottom view of the therapy pack of FIG. 1 with a re-usable liner cover.

FIG. 3 is a bottom view of the therapy pack of FIG. 2 with the re-usable liner cover removed exposing a gel assembly for re-usable adherence to skin, underneath.

FIG. 4 is an exploded, diagrammatic side view of a layering assembly of the therapy pack of FIG. 3 according to an exemplary embodiment.

FIG. 5 is a perspective, partial top view of the therapy pack of FIG. 3 including a re-usable cover exploded from the pack.

FIG. 6 is a perspective top view of a roll of gel tabs die cut for assembly into a therapy pack system according to an exemplary embodiment.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. Like or similar components are labeled with identical element numbers for ease of understanding.

Referring to the Figures in general, exemplary embodiments of the subject technology provide a therapy pack system 100 that provides hot and/or cold therapy treatment to a human body part with the ability to repeatedly stick directly to the skin, and be repositioned if necessary, after each use. A tab (or greater section of area) includes a sticky or tacky gel polymer element insulating the skin point of contact from direct contact by the therapy pack body. The gel polymer assembly is positioned directly under the therapy pack in between the heat/cold source and the skin providing both a re-usable device for re-adhering to the body and a buffer as the gels don't freeze protecting the skin from the types of burn associated with using hot/cold therapy packs. Embodiments of a gel assembly disclosed below may be one or a variation of combinations that include a single layer of gel polymer coupled to the therapy pack, a gel polymer layer coupled to a non-woven fabric structure including an acrylate adhesive on one or both sides of the non-woven fabric, or a double layer of gel polymer. Another option may include applying acrylate to the underside tie layer of the gel directly, without use of a non-woven.

In comparison, conventional hot or cold therapy packs are manually held to the body and are applied directly to the skin of the area being treated. This approach causes a cold pack to heat up prematurely as the hand holding the pack warms up the pack's internal gel. In addition, when initially applied, a conventional ice pack can cause freezer burn or general discomfort to the skin from the cold source being in direct contact. A heat pack may cause heat burn from direct contact to the skin and may be generally too hot to hold in place as the heat released is indiscriminate in the direction emitted. Moreover, other devices in the general field of self-adhering ice packs are single treatment use devices including an adhesive that is not re-usable. If an adhesive is used, the adhesive is provided flanking the section providing therapy similar to a bandage with adhesive wings to avoid damaging the adhesive. However, using an adhesive with an ice pack is unlikely to have desirable results because conventional adhesives are prone to freezing and breaking down causing the attachment to the skin to fail which will also preclude an adhesive from being reused with the ice pack.

It will be appreciated however that aspects of the disclosed therapy pack system allow the therapy pack to conform to the body part, (in some cases being able to wrap around the contours of the treated part) while self-adhering to the person. The gel polymer assembly holds the therapy pack in place without assistance. Hands are thus protected from discomfort because they are no longer needed to hold the pack in place. The gel polymer further protects the treated area from direct contact with the hot/cold source by providing a buffer between the skin area of attachment and the pack. Moreover, the gel polymer assembly is re-usable so that the same therapy pack may be applied repeatedly after the heat/cold source wears off and needs refreshing.

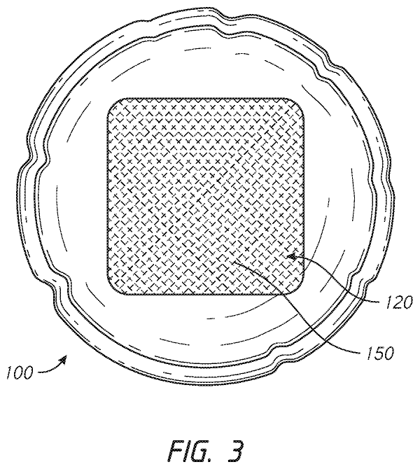

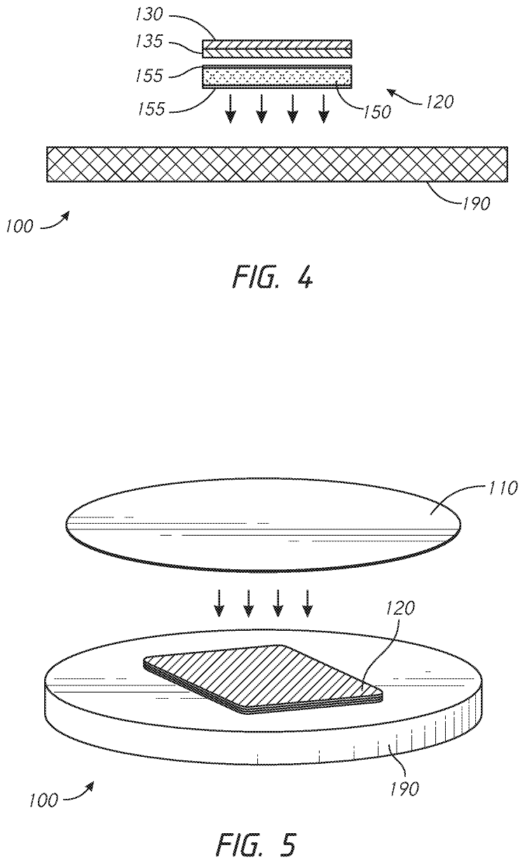

Referring now to FIGS. 1-5, an exemplary embodiment of the therapy pack system 100 is shown. In general, the system 100 includes gel die tab assembly 120 coupled to a therapy pack 190. The therapy pack 190 may be a hot and/or cold based gel housed within a polymer casing 105. In some embodiments, a hybrid pack material may be used for the casing 105 to keep the pack malleable while cold. For example, the casing 105 may be made from a corona treated, polymer such as a tri-polyester pack film. This allows for a “low energy” surface to be printed. Additionally, the corona treated material gives additional surface roughness to mate to corona treated surfaces of the gel die tab assembly 120 in some embodiments. The gel in each casing 105 may be a mixture of water, hydroxymethyl cellulose, and glycerin to a specified amount.

The gel die tab assembly 120 may comprise a medical grade gel polymer 130. The gel polymer 130 is sticky or tacky and holds the therapy pack 190 to the skin without the need for a person to manually hold the therapy pack 190 in place. In addition, the gel polymer 130 retains its tackiness so that the gel die tab assembly 120 is re-usable to re-attach the therapy pack 190 as needed for several uses. In an exemplary embodiment, the gel polymer 130 is a hydrogel. In some embodiments, the gel polymer 130 may be directly mounted onto the exterior surface of the casing 105. In embodiments using gel polymer 130 directly mounted onto the casing 105, the gel polymer 130 may be for example, 32-45 mil thick. This allows greater distance between skin and the therapy pack 190. Testing may indicate that a thicker gel polymer 130 layer is better, with regard to safety. In addition, the gel polymer 130 is highly flexible providing greater indexing to contours of the body.

In order for the gel polymer 130 to not freeze, or come to a core temperature that would decrease its usability, some embodiments may mix a salt (for example, sodium chloride) in to the gel. Since ice naturally repels salt water molecules, the gel polymer 130 freezing point is lower than the freezing point of water. The content of chloride in the gel solution is proprietary, however, it may range from 3-13% chloride by volume. At this level, freezers would need to be set for example to 24 degrees Fahrenheit or lower to begin to freeze the water molecules.

In some embodiments, the gel polymer 130 layer may be layered onto a non-woven fabric support structure 150. In some embodiments, the gel polymer 130 may be impregnated into the non-woven fabric support layer 150 so that the interstices of the non-woven fabric are filled to a depth that is about half way through the fabric layer but may stop short of reaching the opposite side of the fabric in embodiments that may include an adhesive backside (described further below) so as not to contaminate the adhesive. The non-woven fabric support structure 150 may be for example, a rigidizer or a spunbond polypropylene. The non-woven fabric may be a patch. In an exemplary embodiment, the patch of non-woven fabric (or the gel polymer if the non-woven fabric is omitted) has a perimeter less than the area of the casing 105. As will be appreciated, the therapy pack 190 is permitted to wrap around body contours while the gel assembly 120 maintains the system 100 in place. When the gel polymer 130 is layered onto the support structure 150, the thickness of the gel polymer may be approximately 22-40 mils thick.

Some embodiments may use one layer of gel polymer 130 on top of another layer of gel polymer 130. The two layers of gel polymer 130 may in aggregate, provide a thickness of about 50-55 mils. One or more tie layers may be sandwiched between the two layers of gel polymer 130. By raising the thickness of the gel polymer in the system 100, the outermost layer 130 is less likely to freeze (and thus, be re-usable over a longer period of time/number of uses) while still providing the buffer zone to protect the skin from heat or freezer burn. By using a doubled layer of gel polymer 130, the non-woven fabric support 150 may be omitted. As will be appreciated, this may provide significant savings in manufacturing costs since processes related to producing the non-woven fabric support structure 150, corona treating the non-woven fabric layer, adding adhesive to the non-woven fabric and coupling the non-woven fabric layer 150 to the gel polymer 130 is avoided. In an alternate embodiment, a single gel polymer layer 130 may be manufactured according to a custom mold with a thickness of approximately 50-55 mils. This range is generally thicker than industry standard for gel dies. However, the thickness provides both the buffer zone and resistance to freezing desired from the gel polymer 130 for aspects of the subject technology. Some embodiments may use a 40 mil thick gel polymer layer 130 and paint acrylate applied directly to the underside of the gel polymer layer 130 omitting the non-woven fabric support layer 150 with the gel polymer layer 130 having support from a tie layer, which is typically already a non-woven type armature present.

In embodiments using a non-woven fabric support 150, an acrylate adhesive layer 155 may be positioned between the exterior surface of the casing 105 and the pack side of the non-woven fabric support 150. Some embodiments may also include a second layer of acrylate adhesive 155 in between the gel polymer 130 and the skin side of the support structure 150. A tie layer 135 which may be for example, a co-extruded poly vinyl or a polyolefin or nylon, may be sandwiched between the gel polymer 130 and the second layer of acrylate 155. As will be appreciated, the undersides' acrylate adhesive layer(s) 155 help insulate the gel's tie layer 135 and serves to better secure the gel polymer 130 to the therapy pack 190. In some embodiments, the non-woven fabric support structure 150 may be corona treated on one or both sides to improve the receipt of the adhesive layer(s) 155. As will be appreciated, the higher the number of different layers used, the better the performance in insulating the skin from the therapy pack 190 cold/heat source.

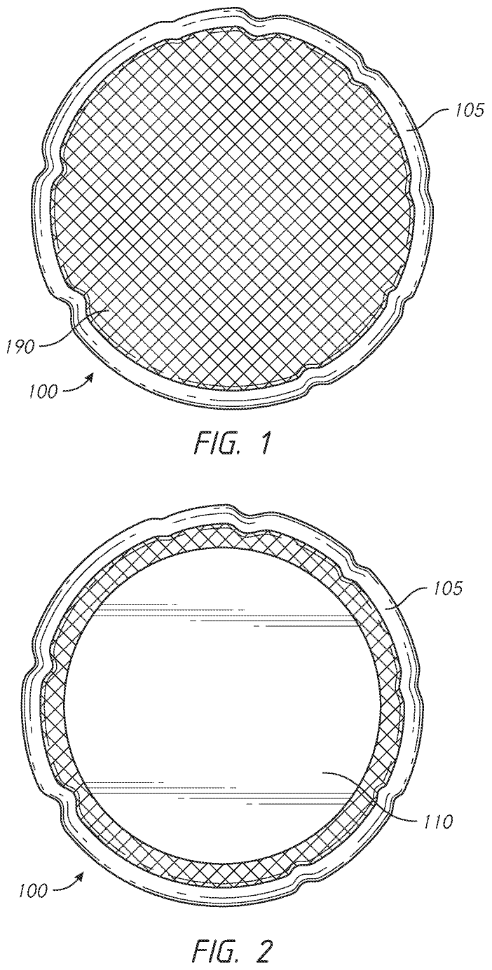

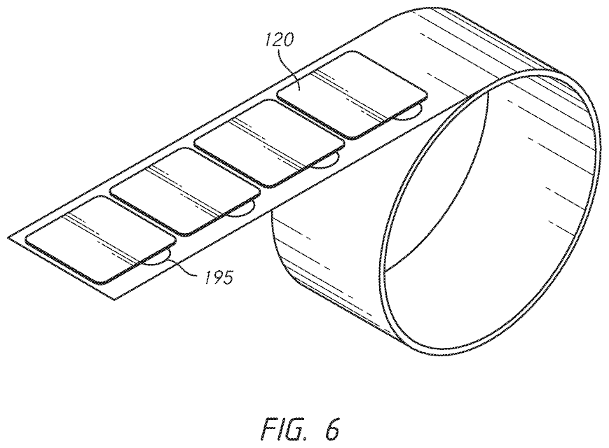

The exposed side of the gel polymer 130 may be protected by a siliconized, replaceable and releasable PET release liner (sometimes referred to as “cover”) 110 (shown in FIGS. 2 and 5). The PET release liner 110 may be approximately 5 mil thick and in some embodiments includes a tab for the user to grasp. The cover 110 may be a non-tear-able polyester that is coated with silicone at a proprietary release value. Silicone acts as a “temperature” block for the gel polymer 130 it covers. It protects the gel polymer 130, adding to shelf life, and also allows the gel polymer 130 to be reused. While many release liners are normally considered a “throw-away” component, the cover 110 of the subject technology is re-usable and releasable from the gel assembly 120 for several (for as long as the gel is active) uses. In an exemplary embodiment, the cover 110 includes an exterior side of treated polyester made porous, printed, UV coated and die cut to shape. An interior, pack side includes a silicone and wax mix layer. The proprietary mix allows the manufacturer to control exact adhesion rates.

As is known in the industry, release liners require “other” liners to applied in order to be registered, printed and die cut. That creates a problem in production in that the manufacturer has to remove a liner from the release liner cover. This requires machinery or a time consuming process of using your fingers/nails to remove each one. Referring now to FIG. 6, the applicant has discovered a solution to address this issue. When die cut, the supplier adds a piece of weld-tape 195 to the units of gel adhesive assemblies 120. The pull pressure is exactly 3 lbs. This allows the gel adhesive assembly 120 tab to be pulled back without the tape 195 coming off prematurely, revealing the “cover,” while not disrupting or removing any treated surface.

As will be appreciated from the foregoing, the non-woven support structure 150 acts as a “barrier,” providing the correct amount of cold/heat therapy w/o tissue injury. The therapy pack 190 body may be made of a polyester/polyamide (PE/PA) material which is not your typical PVC that is frequently used with low-cost round ice packs. PE/PA material has a low-energy surface and uses an ionic surface treatment to allow the multi-component gel assembly 120 to attach to the pack 190 casing 105. As will be appreciated, PE/PA is also more flexible and reliable at freezing or sub-freezing temperatures. The adhesive gel assembly 120 may be re-frozen/re-heated for re-use and the cover liner 110 may be removed to expose the adhesive gel assembly 120 and replaced for use again in a subsequent therapy session.

As will be further appreciated, the therapy pack system 100 has a low profile providing ease of bend conformity while simultaneously allowing it to adhere around limb surfaces. When using hydrogel, the hydrogel is 90% water and is hydrophilic. The total therapy pack system 100 thickness is approximately 22-55 mil (depending on the embodiment used), the approximate equivalent of 2.8-3.4 sheets of paper towel cloth that one would normally put between a conventional ice pack and skin. In embodiments using a single acrylate layer 155, moisture that is created upon cool-down of the pack is allowed to escape through the non-woven fabric layer 150 and wick into the hydrophilic hydrogel layer 130, thereby extending the life of the adhesion. There is less wicking as a result of the additional acrylate layer 155 and having adhesive on both sides of the (non-woven) layer 150 further limits the wicking. With acrylate adhesive, there is 10% wicking while without the presence of acrylate adhesive, there is up to 80% wicking. The tradeoff is the gel adhesive 130 sticks to the skin with better adhesion.

As will be appreciated, the therapy pack system 100 sticks to clean, dry skin and is repositionable. The tensile strength of hydrogel is approximately 240 grams per inch of width (on skin). In some embodiments, the hydrogel is specially made for this product. As shown in FIG. 6, the pack components may be manufactured into a roll. Rolls of gel may be slit, delaminated, are impregnated on rolls of non-woven PP material and rotary die cut into parts to form the gel assemblies 120. The adhesive part is then applied to the pack surface 190 (FIG. 1) directly from the roll.

Those of skill in the art would appreciate that various components and blocks may be arranged differently (e.g., arranged in a different order, or partitioned in a different way) all without departing from the scope of the subject technology.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

What is claimed is:

1. A self-adhering therapy pack system, comprising:
a hot/cold therapy pack comprising a gel housed in a corona treated polymer casing adapted for use in hot or cold type therapy, wherein the gel is configured to be frozen for cold therapy applications and heated for heat therapy applications;
a first layer of acrylate adhesive, wherein a first side of the first layer of acrylate adhesive is coupled to an exterior surface of the polymer casing;
a layer of polyester rigidizer non-woven fabric, wherein a first side of the layer of polyester rigidizer non-woven fabric is attached to a second side of the first layer of acrylate adhesive;
a second layer of acrylate adhesive positioned on a second side of the layer of polyester rigidizer non-woven fabric, wherein the layer of polyester rigidizer non-woven fabric is positioned between the first layer of acrylate adhesive and the second layer of acrylate adhesive;
a 51-55 mil think layer of tacky adhesive hydrogel attached to a skin facing side of the second layer of acrylate adhesive and positioned for direct contact with skin of a user, wherein:
the layer of adhesive hydrogel is positioned under the gel housed in the polymer casing and between the polymer casing and skin of the user when the system is in use,
the layer of adhesive hydrogel comprises 3%-13% chloride by volume, and
the layer of adhesive hydrogel is configured to be re-frozen and re-heated and remain tacky for a plurality of re-uses; and
a polyester cover re-attachable to the layer of adhesive hydrogel and re-usable to cover the layer of adhesive hydrogel.

2. The self-adhering therapy pack system of claim 1, wherein the layer of polyester rigidizer non-woven fabric is corona treated on a front and back side of the fabric.

3. The self-adhering therapy pack system of claim 1, wherein the layer of hydrogel is a tab covering an area less than a perimeter of the polymer casing.

4. The self-adhering therapy pack system of claim 1, further comprising a topical agent impregnated into the hydrogel.

5. The self-adhering therapy pack system of claim 1, further comprising a layer of spunbound polypropylene, non-woven fabric positioned between the exterior surface of the polymer casing and the layer of adhesive hydrogel.

6. The self-adhering therapy pack system of claim 1, wherein the hot/cold therapy pack is configured to conform to a body part onto which the system is removably attached.
